(19) United States
(12) Patent Application Publication
(10) Pub. No.: US 2010/0207038 A1
(43) Pub. Date: Aug. 19, 2010

(54) Apparatus and method for laser irradiation

(75) Inventors: Barry Leonard Sharp, Leicestershire (GB); Dhinesh Asogan, Leicestershire (GB); Ciaran John Patrick O'Connor, Cambridgeshire (GB)

Correspondence Address: HARRINGTON & SMITH, 4 RESEARCH DRIVE, Suite 202, SHELTON, CT, 06484-6212, US

(73) Assignee: Loughborough University

(21) Appl. No.: 12/378,458

(22) Filed: Feb. 13, 2009

(51) Int. Cl. A61N 5/00; G21G 4/00

(52) U.S. Cl. 250/492.1; 250/493.1

(57) ABSTRACT

An apparatus for laser irradiation including a housing wherein the housing defines at least a portion of a test volume; a first conduit configured to provide a first flow of gas into the test volume; a second conduit configured to remove the first flow of gas from the test volume; a third conduit configured to provide a second flow of gas; and a flow controller configured to provide a region of increased flow impedance between the first conduit and the third conduit so that the first flow of gas is directed towards the test volume and the second flow of gas is directed away from the first flow of gas so as to restrict gas external to the housing from coming into contact with the first flow of gas.

### FIELD OF THE INVENTION

Embodiments of the present invention relate to an apparatus and method for laser irradiation. In particular, they relate to an apparatus and method for controlling the environment in which laser irradiation takes place.

### BACKGROUND TO THE INVENTION

Laser ablation is a method in which a sample is irradiated with a laser beam such that material is removed from the sample. Laser ablation has a wide range of uses in many different areas of technology. For example it may be used to remove material for analysis so that the chemical or structural composition of a sample may be determined. A laser may also be used to cut or drill into a sample or to scribe or etch into the surface of a sample. Laser ablation may also be used to remove any unwanted material from a sample, this may be useful for removing unwanted short circuits in an electrical circuit or for cleaning a sample by removing any impurities from the surface of the sample.

Matrix assisted laser desorption ionization (MALDI) is a method in which an analyte is held within a matrix. The matrix is then irradiated with a laser and absorbs the energy from the laser. Some of the energy from the laser is then transferred to the analyte so that the analyte is ionized and removed from the sample. The matrix enables energy to be transferred from the laser to the analyte.

In many applications of laser irradiation such as laser ablation and MALDI it is advantageous to be able to control the environment in which the laser ablation or MALDI takes place. For example, it may be advantageous to prevent the sample from coming into contact with ambient air as this may oxidize the sample or contaminate the material which has been removed from the sample.

Known methods of controlling the environment in which laser ablation or MALDI takes place involve placing the sample within a sealed container, however this limits the size of the samples which can be used as they must be able to fit inside the container. In some known methods a relatively large gas flow may be required to control the environment within the container.

### BRIEF DESCRIPTION OF VARIOUS EMBODIMENTS OF THE INVENTION

According to various, but not necessarily all, embodiments of the invention there is provided an apparatus for laser irradiation comprising: a housing wherein the housing defines at least a portion of a test volume; a first conduit configured to provide a first flow of gas into the test volume; a second conduit configured to remove the first flow of gas from the test volume; a third conduit configured to provide a second flow of gas; and a flow controller configured to provide a region of increased flow impedance between the first conduit and the third conduit so that the first flow of gas is directed towards the test volume and the second flow of gas is directed away from the first flow of gas so as to restrict gas external to the housing from coming into contact with the first flow of gas.

Embodiments of the invention provide the advantage that the second flow of gas is used to block the ingress of other gases into the first flow of gas and the test volume where the laser irradiation is taking place. This prevents the sample from coming into contact with ambient air or other gases external to the housing so it is not necessary to place the apparatus in a sealed box or to create a vacuum around the apparatus.

Also the use of a flow controller between the first conduit and the third conduit ensures that the first flow of gas may be independent from the second flow of gas. This enables the amount of gas flow required to control the laser irradiation environment to be reduced.

In some embodiments of the invention the apparatus may comprise an optical transmitter configured to enable a laser to be transmitted into the test volume. The optical transmitter may be, for example, an optical window or an optical fibre.

In some embodiments of the invention the apparatus may comprise a plurality of second conduits configured to remove the first flow of gas from the test volume. Such embodiments of the invention provide the advantage that the flow of gas from each of the second conduits may be provided to a different output. For example, the flow from one conduit may be provided to a first type of analytical apparatus such as an inductively coupled plasma mass spectrometry (ICP-MS) apparatus or MALDI mass spectrometer while the flow from another conduit may be provided to a different type of analytical apparatus such as an apparatus for inductively coupled plasma optical emission spectrometry (ICP-OES) or any other suitable type of optical detector.

In some embodiments of the invention a jet pump may be coupled to at least one second conduit. Such embodiments of the invention provide the advantage that the jet pump enables the proportion of gas from the first flow of gas which enters the test volume to be controlled.

In some embodiments of the invention the first conduit may comprise a grating. In some embodiments of the invention the third conduit may comprise a grating. Such embodiments of the invention reduce the amount of turbulence in the flows of gas.

In some embodiments of the invention the apparatus comprises an electrical connection configured to provide an electrical charge to the grating.

In some embodiments of the invention the flow controller may be coupled to the grating.

In some embodiments of the invention the gas removed from the test volume may comprise particles of solid or liquid dispersed within the flow of gas.

According to various, but not necessarily all, embodiments of the invention there is provided a method of laser irradiation comprising: positioning a housing over a sample wherein the housing defines at least a portion of a test volume; directing a laser beam toward the sample; directing a first flow of gas into the test volume; removing the first flow of gas from the test volume; and directing a second flow of gas away from the first flow of gas so that the second flow of gas restricts gas external to the housing from coming into contact with the first flow of gas.

In some embodiments of the invention the laser beam may be directed through an optical transmitter into the test volume.

In some embodiments of the invention a plurality of conduits may be provided for extracting gas from the test volume. In some embodiments of the invention a jet pump may be used to extract gas from the test volume.

In some embodiments of the invention the method may also comprise directing the first flow of gas through a grating. In some embodiments of the invention the method may also comprise directing the second flow of gas through a grating. In some embodiments of the invention an electrical charge may be provided to the grating.

In some embodiments of the invention a flow controller may be coupled to the grating and configured to direct the first flow of gas into the test volume and direct the second flow of gas away from the first flow of gas.

In some embodiments of the invention the first flow of gas may comprise helium or argon. In some embodiments of the invention the second flow of gas may comprise nitrogen or argon.

According to various, but not necessarily all, embodiments of the invention there is provided an apparatus comprising: a housing wherein the housing defines at least a portion of a test volume; a first conduit configured to provide a first flow of gas into the test volume; a second conduit configured to remove the first flow of gas from the test volume; a third conduit configured to provide a second flow of gas; and a flow controller configured to provide a region of increased flow impedance between the first conduit and the third conduit so that the first flow of gas is directed towards the test volume and the second flow of gas is directed away from the first flow of gas so as to restrict gas external to the housing from coming into contact with the first flow of gas.

According to various, but not necessarily all, embodiments of the invention there is provided a jet pump comprising: an inlet conduit configured to direct a flow of motive fluid into the jet pump wherein the inlet conduit comprises a constriction configured to increase the velocity of the motive fluid; a connector configured to enable the jet pump to be connected to a conduit of a test volume of a laser irradiation apparatus such that gas extracted from the test volume of the laser irradiation apparatus is drawn into the jet pump; and an outlet conduit configured to discharge the motive fluid and the gas extracted from the test volume of the laser irradiation apparatus from the jet pump.

In some embodiments of the invention the constriction may comprise a sapphire nozzle. In some embodiments of the invention the nozzle may have a diameter of 160 micrometres.

In some embodiments of the invention the outlet conduit of the jet pump may comprise a first portion and a second portion where the first portion has a narrower diameter than the second portion and is configured to prevent gas flowing from the outlet conduit towards to the inlet conduit.

### BRIEF DESCRIPTION OF THE DRAWINGS

For a better understanding of various examples of embodiments of the present invention reference will now be made by way of example only to the accompanying drawings in which:

FIG. 1 is a schematic illustration of a cross section of an apparatus according to embodiments of the invention;

FIG. 2 is a perspective view of an apparatus according to an embodiment of the invention in which the grating and flow controller are visible; and

FIG. 3 is a schematic illustration of a jet pump according to embodiments of the invention.

### DETAILED DESCRIPTION OF VARIOUS EMBODIMENTS OF THE INVENTION

The Figures illustrate an apparatus 1 for laser irradiation comprising: a housing 3 wherein the housing 3 defines at least a portion of a test volume 5; a first conduit 11 configured to provide a first flow of gas into the test volume 5; a second conduit 13 configured to remove the first flow of gas from the test volume 5; a third conduit 15 configured to provide a second flow of gas; and a flow controller 17 configured to provide a region of increased flow impedance between the first conduit 11 and the third conduit 15 so that the first flow of gas is directed towards the test volume 5 and the second flow of gas is directed away from the first flow of gas so as to restrict gas external to the housing 3 from coming into contact with the first flow of gas.

In the following description, unless expressly stated otherwise, the words “connect” and “couple” and their derivatives mean operationally connected or operationally coupled. It is to be appreciated that any number or combination of intervening components can exist including no intervening components.

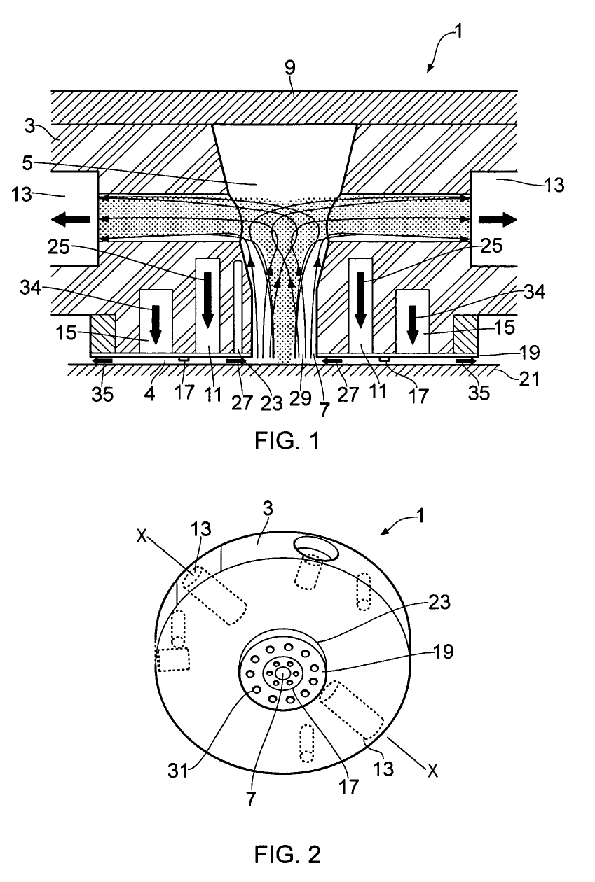

FIG. 1 schematically illustrates a cross section through an apparatus 1 for laser irradiation according to an embodiment of the invention. The cross section is taken through the line X-X as indicated in FIG. 2. FIG. 2 is a perspective view of the apparatus in which the grating 19 and flow controller 17 are visible.

The apparatus 1 may be configured to enable a method such as laser ablation to be used for any of a plurality of different purposes, including, but not limited to, analysis of a sample, cutting of a sample, scribing or etching of a sample or repair of samples such as electronic circuits. In other embodiments of the invention the apparatus 1 may be configured for MALDI.

The apparatus 1 comprises a housing 3. The housing is configured to overlay a sample 21 so that, in the illustrated embodiment, a gap 4 is provided between the housing 3 and the sample 21. In some embodiments of the invention some portions of the housing 3 may contact the sample 21. The width of the gap 4 may be up to 1 mm. The width of the gap 4 used may depend upon the application of the laser irradiation and the gas flows being used.

The housing 3 defines at least a portion of a test volume 5. In the illustrated embodiment the test volume 5 is funnel shaped, that is, it comprises a truncated cone portion and a cylindrical portion at the narrower end of the cone. Other shapes of test volume 5 maybe used in other embodiments of the invention.

The test volume 5 is not sealed and comprises an opening 7 at the narrow end. The opening 7 enables a first flow of gas to enter to the test volume 5. The laser may also be directed through the opening 7 onto the sample 21.

In the illustrated embodiment an optical window 9 is provided at the wider end of the test volume 5. The optical window 9 is configured to enable a laser to be transmitted into the test volume 5. It is to be appreciated that in other embodiments of the invention other optical transmitters may be used, for example, an optical fibre.

The optical window 9 seals this end of the test volume 5 and prevents gas external to the housing 3 from entering the test volume 5 but enables a laser to enter the test volume 5. The optical window 9 may comprise a means for focussing the laser such as a lens.

The apparatus 1 also comprises a first conduit 11. The first conduit 11 is configured to provide a first flow of gas into the test volume 5. In the illustrated embodiment the first conduit 11 is annular and extends around the test volume 5. The first conduit 11 is configured such that, in use, a flow of gas from the first conduit 11 will be directed towards to the test volume 5.

At least one second conduit 13 is provided. The second conduit 13 extends from the test volume 5 and out of the housing 3 and enables the first flow of gas from the first conduit 11 to be removed from the test volume 5. The flow of gas through the second conduit 13 may comprise solid or liquid particles dispersed within the flow of gas. In some embodiments of the invention the flow of gas may comprise an aerosol.

In the illustrated embodiment two second conduits 13 are provided. The two second conduits 13 are provided in the side walls of the test volume 5. In the illustrated embodiment the two second conduits 13 are located at the same height within the test volume 5 and are positioned diametrically opposite each other. It is to be appreciated that in other embodiments of the invention more than two second conduits 13 may be provided and these may be arranged around the test volume 5 in any suitable configuration. Similarly, in other embodiments of the invention there may only be one second conduit 13 provided.

The second conduits 13 may be coupled to another apparatus so that the gas which has been extracted from the test volume 5 is provided to the another apparatus. For example, in embodiments of the invention where laser ablation is being used to analyse the sample 21 the second conduit 13 may provide the gas to an apparatus for analysing the material removed from the sample 21. The apparatus for analysing the sample 21 may be any means which enables the structure or composition of the sample 21 to be determined for example, a mass spectrometer such as an ICP-MS instrument or an apparatus for ICP-OES.

In some embodiments of the invention the second conduit 13 may provide the gas to a control means which may then provide feedback to control the laser. The control means may be any means which is configured to detect an event and, in response to the detection of the event, provide feedback to control the laser. Such events may be, for example, the detection of a material or type of material in the gas flowing through the second conduit 13 or the detection that a certain volume of gas has passed through the second conduit 13.

In some embodiments of the invention the material which may be removed from the sample 21 by the laser may be toxic or harmful, in such cases the second conduits 13 may enable the toxic or harmful fumes to be safely removed.

In embodiments where a plurality of second conduits 13 are provided the second conduits 13 may provide the gas to a plurality of different apparatus.

The apparatus 1 also comprises a third conduit 15 which is positioned adjacent to the first conduit 11 and is configured to provide a second flow of gas. In the illustrated embodiment the third conduit 15 is annular and extends around the first conduit 11. The third conduit 15 is configured so that, in use, the second flow of gas is directed away from the test volume 5.

A flow controller 17 is provided between the first conduit 11 and the third conduit 15. The flow controller 17 extends around the perimeter of the first conduit 11. In the illustrated embodiment the flow controller is a ring which projects out of the surface of grating 19 so that, in use, the flow controller 17 is the closest part of the apparatus 1 to the sample 21. The flow controller 17 increases the impedance to gas flow between the first conduit 11 and the third conduit 15 because the gap 4 between the flow controller 17 and the sample 21 is smaller than the gap 4 between the sample and other parts of the housing 3. By positioning the flow controller 17 between the first conduit 11 and the third conduit 15 the flow of gas from the first conduit 11 is directed in an opposite direction to the flow of gas from the third conduit 15.

It is to be appreciated that in other embodiments of the invention other configurations of flow controller 17 may be used. For example the flow controller 17 may have a different shape or there may be more than one flow controller 17.

The apparatus 1 also comprises a grating 19. In the illustrated embodiment the grating 19 extends over the first conduit 11 and the third conduit 15 so that the flows of gas from the two conduits 11, 15 pass through the grating 19. As can be seen in FIG. 2, in the illustrated embodiment the grating 19 comprises a single annular disc which extends over both the first conduit 11 and the third conduit 15. In other embodiments of the invention a different grating 19 may be provided for each of the different conduits 11, 15.

The grating 19 comprises an array of small holes 31. The holes 31 divide the flows of gas from the conduits 11, 15 from a single large jet into a plurality of smaller jets. This makes the flows of gas quasi-laminar and less turbulent. As the flows of gas are less turbulent this decreases the amount of gas external to the housing 3 which is drawn into the flows of gas and reduces the amount of gas necessary to control the laser irradiation environment.

In the illustrated embodiment the holes 31 only cover a small proportion of the surface area of the grating 19. For example, in some embodiments of the invention the holes 31 may cover approximately 1 percent of the surface area. In other embodiments of the invention the holes 31 may cover a different percentage of the surface area. Covering only a small proportion of the surface area of the grating 19 with holes provides an improved performance of the grating 19.

In some embodiments of the invention the grating may be made from a metal such as nickel.

In the illustrated embodiment the flow controller 17 is attached to the grating 19. This makes the apparatus 1 simple to assemble as it reduces the number of parts which are required. In other embodiments of the invention more than one grating 19 may be provided and the flow controller 17 may be attached either to one of the gratings 19 or directly to the housing 3.

In some embodiments of the invention an electrical connection 23 may be provided to the grating 19. The electrical connection 23 may provide an electrical charge to the grating 19 so that, in use, the grating 19 is electrically biased with respect to the sample 21. This may enable ions which have been removed from the sample 21 by the laser to escape the surface of the sample 21 more easily.

When the apparatus 1 is in use the housing 3 is positioned over a sample 21 so that the test volume 5 overlays the sample 21. The sample 21 may be any item from which material is to be removed by a laser. For example the sample 21 may be a piece of material which is to be analysed or it may be an item such as an electrical circuit which is to be repaired.

In the illustrated embodiment the housing 3 is positioned close to but not touching the sample 21 so that there is a gap 4 between the housing 3 and the sample 21. As mentioned above, the gap 4 between the housing 3 and the sample 21 may be up to 1 mm. The narrowest part of the gap 4 is the point between the flow controller 17 and the sample 21. In other embodiments of the invention the housing 3 may contact the sample 21.

A laser is directed through the optical window 9 and the test volume 5 to the sample 21. The laser causes material to be removed from the sample 21. The material is then extracted from the sample by the gas flow through the test volume 5 as will be described below.

A first flow of gas is provided through the first conduit 11. In the illustrated embodiment the flow of gas through the first conduit 11 is provided downwards towards the sample 21 in the direction indicated by the arrows 25.

The gas used in the first flow of gas may depend on the application of the laser irradiation. In some embodiments of the invention the gas used may be helium. Helium may be a suitable gas to use because it is inert so it will not contaminate the sample 21 or the material removed from the sample 21. Helium also has a low density which makes it easier for material to escape the surface of the sample 21 and a high thermal conductivity which prevents the build up of heat which may affect the efficiency of the laser irradiation. Helium also has a high ionization potential which prevents the gas from forming a plasma which might scatter the laser and affect the nature of the laser interaction with the sample 21. In other embodiments of the invention other gases may be used such as argon.

The first flow of gas through the first conduit 11 may be controlled by a mass flow controller or any other apparatus which enables the flow of gas to be accurately controlled.

In some embodiments of the invention the first conduit 11 may be configured so that the first flow of gas enters the first conduit 11 tangentially. This ensures a good distribution of gas around the perimeter of the first conduit 11 so that there is an even distribution of gas through the grating 19.

The first flow of gas flows through the grating 19 and into the gap between the sample 21 and the housing 3. The first flow of gas is then directed towards the test volume 5 by the flow controller 17 so that the first flow of gas flows along the gap 4 to the test volume 5 in the direction of arrows 27.

The first flow of gas then enters the test volume 5 through the opening 7 where the material removed from the sample 21 by the laser is entrained within the first flow of gas. The first flow of gas then flows upwards through the test volume 5 toward the second conduits 13 as indicated by the arrows 29 in FIG. 1.

The first flow of gas and the material entrained within the gas then flows though the second conduits 13 and so is removed from the test volume 5. As described above, the second conduits 13 may be coupled to other apparatus so that the flow of gas is provided to other apparatus.

A second flow of gas is provided through the third conduit 15. In the illustrated embodiment the flow of gas through the third conduit 15 is provided downwards towards the sample 21 in the direction indicated by the arrows 34. The second flow of gas through the third conduit 15 is parallel to the first flow of gas through the first conduit 11.

The gas used in the second flow of gas may depend on the application of the laser irradiation. In some embodiments of the invention the gas used may be nitrogen as this may provide cost benefits. In some embodiments of the invention the gas used in the second gas flow may be argon as this may also be used for ICP-MS.

The second flow of gas through the third conduit 15 may be controlled by a mass flow controller or any other apparatus which enables the flow of gas to be accurately controlled.

In some embodiments of the invention the third conduit 15 may be configured so that the second flow of gas enters the third conduit 15 tangentially. This ensures a good distribution of gas around the perimeter of the third conduit 15 so that there is an even distribution of gas through the grating 19.

The second flow of gas flows through the grating 19 and into the gap 4 between the sample 21 and the housing 3. The second flow of gas is then directed in the opposite direction to the first flow of gas by the flow controller 17 so that the second flow of gas flows along the gap 4 away from the test volume 5 and towards the edge of the housing 3 as indicated by the arrows 35.

The second flow of gas acts as a barrier to the first flow of gas and prevents other gases, external to the apparatus, from coming into contact with the first flow of gas. The apparatus 1 may be configured so that only the gas from the first conduit 11 enters the test volume 5. This enables the environment in the test volume 5 to be controlled so that it is suitable for the laser irradiation. Using a second flow of gas to prevent external gases from entering the test volume 5 means that it is not necessary to place the apparatus 1 in a sealed container so the apparatus 1 may be used for any size of sample 21.

Also the two flows of gas maybe independently controlled which means that the overall gas flow may be reduced.

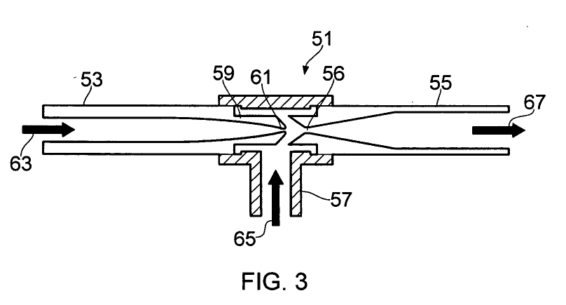

In some embodiments of the invention a jet pump 51 may be connected to one or more of the second conduits 13. An example of a jet pump 51 which may be used in embodiments of the invention is illustrated in FIG. 3.

The jet pump 51 comprises an inlet conduit 53, an outlet conduit 55 and a connector 57.

The inlet conduit 53 is configured to be connected to a supply of motive fluid at one end and connected to the jet pump 51 at the other end.

The inlet conduit 53 comprises a constriction 59 at the end connected to the jet pump 51. At the constriction 59 the cross sectional area of the inlet conduit 53 is gradually decreased. The constriction 59 terminates in a nozzle 61. The nozzle 61 may have a very small diameter. In some embodiments of the invention the diameter of the nozzle 61 may, be approximately 160 micrometers.

The nozzle 61 may be made of any suitable material for example sapphire.

The outlet conduit 55 is configured to be connected to the jet pump at one end. The second end may be connected to another apparatus such as an ICP-MS apparatus.

The outlet conduit 55 is positioned relative to the inlet conduit 53 so that the gas flow from the inlet conduit 53 flows into the outlet conduit 55.

The portion of the outlet conduit 55 connected to the jet pump 51 comprises a narrow throat 56 which has a small cross sectional area. In the illustrated embodiment the cross sectional area of the outlet conduit 55 gradually increases from the narrow throat 56 until the cross sectional area of the outlet conduit 55 is greater than the cross sectional area of the inlet conduit 53. It is to be appreciated that in other embodiments of the invention the outlet conduit 55 and the inlet conduit 53 may have the same cross sectional area or the inlet conduit 53 may have a greater cross sectional area than the outlet conduit 55.

The connector 57 is configured so that one end may be connected to a second conduit 13 of the laser irradiation apparatus 1 and the other end may be connected to the jet pump 51. The connector 57 enables the flow of gas removed from the test volume 5 to be provided to the jet pump 51.

In use a motive fluid is provided to the jet pump 51 as indicated by the arrow 63. The motive fluid used may depend upon the application of the invention. In some embodiments of the invention the motive fluid used may be argon as this may be used for ICP-MS.

As the motive fluid flows through the constriction 59 the velocity of the fluid increases because the cross sectional area of the inlet conduit is decreasing 59. The jet of fluid which exits inlet conduit at the nozzle 61 may have a very large velocity. In some embodiments of the invention the velocity of the jet may be close to or faster than the speed of sound in the motive fluid.

The fluid leaving the inlet conduit 53 has a very high velocity. The jet pump may be configured so that fluid leaving the inlet conduit 53 has a low pressure region. The low pressure region may be on the axis of symmetry of the jet. This draws the gas from the connector 57 into the jet pump 51 as indicated by the arrow 65. This enables the flow of gas from the test volume 5 to be drawn out of the test volume 5 and into the jet pump 51.

As the jet of fluid exiting the inlet conduit 53 has a very high velocity it is also very turbulent. The turbulence and viscous drag on the surrounding fluid mean that the jet of fluid is strongly entraining which provides another mechanism by which gas from the test volume 5 is drawn out of the test volume 5 and into the jet pump 51. This also enables the gas flowing in through the connector 57 to be efficiently mixed with the motive fluid and carried into the outlet conduit 55. Therefore the jet pump 51 provides an efficient means for mixing the gas from the test volume 5 with another fluid.

The mixture of the motive fluid and the flow of gas from the test volume 5, then enters the outlet conduit 55 where it flows along the outlet conduit 55 away from the jet pump 51 as indicated by the arrow 67.

The narrow throat 56 of the outlet conduit 55 prevents fluid from the outlet conduit from flowing back down the outlet conduit towards the inlet conduit 53. The increasing cross section of the outlet conduit 55 acts as a diffuser and decreases the velocity of the flow of gas in the conduit.

Therefore the jet pump 51 provides a means for controlling the proportion of flow of gas from the first conduit 11 which flows into the test volume 5. This provides an improved control over the environment in which the laser irradiation takes place. The jet pump 51 also enables the gas to be efficiently removed from the test volume 5.

Although embodiments of the present invention have been described in the preceding paragraphs with reference to various examples, it should be appreciated that modifications to the examples given can be made without departing from the scope of the invention as claimed. For example in the above described embodiments a laser is used to provide energy to the sample in order to remove material from the sample. In other embodiments of the invention an energy source other than a laser such as a supersonic aerosol stream or a spark for a conducting sample may be used.

Also in the illustrated embodiments the flow controller 17 increases the impedance to gas flow between the first conduit 11 and the third conduit 15 by decreasing the cross sectional area of the gap 4 between the first conduit 11 and the third conduit 15. It is to be appreciated that other means of providing increased impedance to gas flow may be used in addition to or instead of the reduced cross sectional area of the gap 4. For example, the flow of gas through the first conduit 11 and the third conduit 15 may be controlled so that there is a larger flow of gas through the third conduit 15, the apparatus 1 may be configured so that the impedance to gas flow into the surrounding atmosphere is less than the impedance to gas flow through the second conduit 13, the grating 19 may be configured to control the stiffness and direction of the flow of gas etc.

Features described in the preceding description may be used in combinations other than the combinations explicitly described.

Although functions have been described with reference to certain features, those functions may be performable by other features whether described or not.

Although features have been described with reference to certain embodiments, those features may also be present in other embodiments whether described or not.

Whilst endeavoring in the foregoing specification to draw attention to those features of the invention believed to be of particular importance it should be understood that the Applicant claims protection in respect of any patentable feature or combination of features hereinbefore referred to and/or shown in the drawings whether or not particular emphasis has been placed thereon.

1. An apparatus for laser irradiation comprising:
a housing wherein the housing defines at least a portion of a test volume;
a first conduit configured to provide a first flow of gas into the test volume;
a second conduit configured to remove the first flow of gas from the test volume;
a third conduit configured to provide a second flow of gas; and
a flow controller configured to provide a region of increased flow impedance between the first conduit and the third conduit so that the first flow of gas is directed towards the test volume and the second flow of gas is directed away from the first flow of gas so as to restrict gas external to the housing from coming into contact with the first flow of gas.

2. An apparatus as claimed in claim 1 wherein the apparatus comprises an optical transmitter configured to enable a laser to be transmitted into the test volume.

3. An apparatus as claimed in claim 1 wherein the apparatus comprises a plurality of second conduits configured to remove the first flow of gas from the test volume.

4. An apparatus as claimed in claim 1 wherein a jet pump is coupled to at least one second conduit.

5. An apparatus as claimed in claim 1 wherein the first conduit comprises a grating.

6. An apparatus as claimed in claim 1 wherein the third conduit comprises a grating.

7. An apparatus as claimed in claim 5 comprising an electrical connection configured to provide an electrical charge to the grating.

8. An apparatus as claimed in claim 5 wherein the flow controller is coupled to the grating or the housing.

9. A method of laser irradiation comprising:
positioning a housing over a sample wherein the housing defines at least a portion of a test volume;
directing a laser beam toward the sample;
directing a first flow of gas into the test volume;
removing the first flow of gas from the test volume; and
directing a second flow of gas away from the first flow of gas so that the second flow of gas restricts gas external to the housing from coming into contact with the first flow of gas.

10. A method as claimed in claim 9 wherein the laser beam is directed through an optical transmitter into the test volume.

11. A method as claimed in claim 9 wherein a plurality of conduits are provided for extracting gas from the test volume.

12. A method as claimed in claim 9 wherein a jet pump is used to extract gas from the test volume.

13. A method as claimed in claim 9 further comprising directing the first flow of gas through a grating.

14. A method as claimed in claim 9 further comprising directing the second flow of gas through a grating.

15. A method as claimed in claim 13 comprising providing an electrical charge to the grating.

16. A method as claimed in claim 13 wherein a flow controller is coupled to the grating or the housing and configured to direct the first flow of gas into the test volume and direct the second flow of gas away from the first flow of gas.

17. A method as claimed in claim 9 wherein the first flow of gas comprises helium or argon.

18. A method as claimed in claim 9 wherein the second flow of gas comprises nitrogen or argon.

19. An apparatus comprising:
a housing wherein the housing defines at least a portion of a test volume;
a first conduit configured to provide a first flow of gas into the test volume;
a second conduit configured to remove the first flow of gas from the test volume;
a third conduit configured to provide a second flow of gas; and
a flow controller configured to provide a region of increased flow impedance between the first conduit and the third conduit so that the first flow of gas is directed towards the test volume and the second flow of gas is directed away from the first flow of gas so as to restrict gas external to the housing from coming into contact with the first flow of gas.

20. A jet pump comprising:
an inlet conduit configured to direct a flow of motive fluid into the jet pump wherein the inlet conduit comprises a constriction configured to increase the velocity of the motive fluid;
a connector configured to enable the jet pump to be connected to a conduit of a test volume of a laser irradiation apparatus such that gas extracted from the test volume of the laser irradiation apparatus is drawn into the jet pump; and an outlet conduit configured to discharge the motive fluid and the gas extracted from the test volume of the laser irradiation apparatus from the jet pump.

21. A jet pump as claimed in claim 20 wherein the constriction comprises a sapphire nozzle.

22. A jet pump as claimed in claim 20 wherein the nozzle has a diameter of 160 micrometres.

23. A jet pump as claimed in claim 20 wherein the outlet conduit comprises a first portion and a second portion where the first portion has a narrower diameter than the second portion and is configured to prevent gas flowing from the outlet conduit towards to the inlet conduit.
